(19) United States
(12) Patent Application Publication
(10) Pub. No.: US 2026/0360553 A1
(43) Pub. Date: Nov. 12, 2026

(54) METHOD OF PREDICTING THE ABSOLUTE RISK REDUCTION FROM ANDROGEN DEPRIVATION THERAPY ADDED TO RADIATION THERAPY IN PATIENTS WITH PROSTATE CANCER

(71) Applicant: Myriad Genetics, Inc., Salt Lake City, UT (US)

(72) Inventors: Lauren Lenz, Salt Lake City, UT (US); Alexander Gutin, Salt Lake City, UT (US)

(73) Assignee: Myriad Genetics, Inc., Salt Lake City, UT (US)

(21) Appl. No.: 19/486,873

(22) PCT Filed: May 23, 2024
(86) PCT No.: PCT/US2024/030876

§ 371 Date: Nov. 21, 2025

Related U.S. Application Data
(60) Provisional application No. 63/468,759, filed on May 24, 2023.

(51) Int. Cl. G16H 50/30; G16B 25/10

(52) U.S. Cl. CPC G16H 50/30; G16B 25/10

(57) ABSTRACT

Described herein, are methods of determining an absolute risk reduction (AAR) of disease progression in a subject. Specifically, the present disclosure provides, among other things, methods for determining AAR in a subject receiving androgen deprivation therapy (ADT) and radiotherapy (RT) compared to RT alone where the absolute risk reduction is a measure of the likelihood of metastasis.

### CROSS-REFERENCE TO RELATED PATENT APPLICATIONS

This application claims the benefit of U.S. Provisional Application Ser. No. 63/468,759, filed May 24, 2023. The disclosure of the prior application is considered part of (and is incorporated by reference in) the disclosure of this application.

### TECHNICAL FIELD

Described herein, are methods of determining an absolute risk reduction (AAR) of disease progression in a subject. Specifically, the present disclosure provides, among other things, methods for determining AAR in a subject receiving androgen deprivation therapy (ADT) and radiotherapy (RT) compared to RT alone where the absolute risk reduction is a measure of the likelihood of metastasis.

### BACKGROUND

Approximately one third of males with newly diagnosed localized prostate cancer receives radiation therapy as their primary treatment. Numerous prospectively randomized trials have proven that adding androgen deprivation therapy (ADT) to prostate-directed radiation therapy (RT) improves metastasis-free survival at the population level. Preventing metastasis is a critical goal of therapy, as metastasis is a robust surrogate endpoint for overall survival and is specifically associated with a significant risk of death from prostate cancer. Furthermore, males with metastatic prostate cancer generally rate their overall quality of life as poor.

Combining ADT with RT reduces the risk of metastasis and death, therefore expert consensus guidelines recommend this combination over RT alone for males with unfavorable intermediate-risk or high-risk localized prostate cancer. However, ADT causes adverse effects, including hot flashes, fatigue, sexual dysfunction, bone loss, cognitive changes, and cardiovascular risks. Even when used as a short course (i.e., less than one year), ADT can cause an immediate decrease in quality of life, which can endure for years after its discontinuation.

Patients and physicians can utilize measures like the hazard ratio or relative risk, absolute risk reduction, and number needed to treat (NNT) to compare one therapy against another. These metrics are often derived at the population level for various cohorts of males enrolled in randomized clinical trials.

Most patients would defer using androgen deprivation therapy (ADT) with radiation therapy (RT) when the number-needed-to-treat (NNT) exceeds 25 persons to prevent 1 from developing metastasis, corresponding to an absolute benefit of 4%. However, the magnitude of benefit may differ for individuals within each risk population. Computing an individual's absolute risk-reduction and NNT requires a precise and accurate prognostic estimate of that person's baseline risk. However, no tools for estimating personalized NNT or absolute benefit from ADT exist. One such estimator is a combined clinical cell-cycle risk (CCR) score, which combines clinical components with a molecular cell cycle progression (CCP) component, to estimate the 10-year risk of metastatic disease and prostate cancer-specific mortality. Retrospective analyses have demonstrated the utility of CCR for predicting which patients may benefit from multimodal therapy, RT+ADT. However, no tools for estimating personalized NNT or absolute benefit from ADT exist.

The current disclosure provides a personalized absolute risk reduction and personalized NNT of RT+ADT over RT alone for males with localized prostate cancer receiving prostate-directed RT. The personalized absolute risk reduction from adding ADT to RT is computationally derived by calculating the effect of the trial-established hazard ratio (HR) in males with known CCR scores and metastasis outcomes treated exclusively with RT alone. Knowledge of the personalized absolute risk reduction and personalized NNT for RT+ADT can be a valuable tool in helping patients understand the risks and benefits of using combined modality therapy over RT alone. It may also help in making treatment intensification decisions.

### SUMMARY

One aspect, the present disclosure provides methods of determining an absolute risk reduction (AAR) of disease progression in a subject, comprising: (a) obtaining a biological sample from a subject; (b) calculating a clinical cell-cycle risk (CCR) score (rRT) for treating the subject with radiation therapy (RT) alone; (c) calculating a multimodal risk score (rRT+ADT) for treating the subject with a multimodmodal therapy comprising androgen deprivation therapy (ADT) and RT; and (d) calculating the AAR (rRT−rRT+ADT) for the subject.

In some embodiments, the method further comprises (e) comparing the AAR to a multimodal treatment threshold (MTT), wherein an AAR over the MTT would be predictive of lower risk of metastasis and an AAR under the MTT would be predictive of a higher risk of MTT in subject receiving ADT and RT.

In some embodiments, the CCR score comprises combining a cancer of the prostate risk assessment (CAPRA) score and a cell-cycle progression (CCP) score.

In some embodiments, the CCP score is calculated as the average expression of 31 CCP genes normalized to 15 housekeeping genes.

In some embodiments, the multimodal risk score (r_{RT+ADT}) is calculated according to r_{RT+ADT}=1−(1−r_{RT})^{IIR}_{ADT}, wherein HR_{ADT} is a relative ADT benefit as a hazard ratio.

In some embodiments, the AAR is calculated based on a combination treatment of ADT and RT compared to RT alone.

In some embodiments, the subject has been diagnosed with prostate cancer.

In some embodiments, the disease progression is metastasis.

In some embodiments, the clinical cell-cycle risk (CCR) score (r_{RT}) is a risk estimate of developing metastasis after treatment with RT alone.

In some embodiments, multimodal risk score (r_{RT+ADT}) is a risk estimate of developing metastasis after treatment with ADT and RT in combination.

### BRIEF DESCRIPTION OF THE DRAWINGS

FIGS. 1A, 1i, and 1C show the overview of statistical methods used to model ADT benefit as a function of CCR score. FIG. 1A depicts the calculation of risk of 10-year metastasis with single-mode RT. FIG. 1B depicts the methods to model risk of 10-year metastasis with RT+ADT. FIG. 1C depicts the calculation of absolute risk reduction due to ADT.

FIG. 2 shows the risk of metastasis as a function of CCR score under different assumptions of overall relative benefit of ADT (32%, 41%, and 48%). The vertical dashed line represents the multimodality threshold at CCR=2.112.

FIG. 3 shows the risk of metastasis as a function of CCR score assuming a relative benefit of ADT of 41%, with simulated 95% CIs. The dashed vertical line represents the multimodality threshold at CCR=2.112.

FIG. 4 shows the absolute risk reduction from ADT added to RT with corresponding NNT as a function of CCR score, assuming a relative benefit of ADT of 41%, with simulated 95% CIs. The dashed vertical line represents the multimodality threshold at CCR=2.112.

FIGS. 5A, 5B, and 5C show a more detailed overview of the statistical methods to model ADT benefit as a function of CCR score. FIG. 5A shows the calculation of risk of 10-year metastasis with single-mode RT. FIG. 5B shows the methods to model risk of 10-year metastasis with RT+ADT, including with an interaction between the benefit of ADT and CCR score. FIG. 5C shows the calculation of absolute risk reduction due to ADT.

FIG. 6 depicts the patient benefit from ADT (HR_{ADT}) according to CCR score with no interaction (3=0), 50% maximum interaction strength (13=0.119929), and maximum interaction strength (3=0.2399858).

FIGS. 7A, 7B, and 7C depict the risk of metastasis with simulated 95% CIs as a function of CCR score for different interaction strengths and assuming a relative benefit of ADT of 41%. FIG. 7A depicts the risk of metastasis according to a CCR score with no interaction (β=0). FIG. 7B depicts the risk of metastasis according to a CCR score with 50% maximum interaction strength (β=0.119929). FIG. 7C depicts the risk of metastasis according to a CCR score with maximum interaction strength (13=0.2399858).

FIGS. 8A, 8B, and 8C depict the absolute risk reduction from ADT added to RT as a function of CCR score for different interaction strengths and assuming a relative benefit of ADT of 41%, shown with simulated 95% CIs and corresponding NNT values. FIG. 8A shows the absolute risk reduction from ADT added to RT as a function of CCR score with no interaction (β=0). FIG. 8B shows the absolute risk reduction from ADT added to RT as a function of CCR score with 50% maximum interaction strength (P=0.119929). FIG. 8C shows the absolute risk reduction from ADT added to RT as a function of CCR score with maximum interaction strength (β=0.2399858). The dashed vertical lines represent the multimodality threshold at CCR=2.112.

FIG. 9 shows the predicted 10-year risk of metastasis given single mode RT for patients in the Clinical Cohort by NCCN risk category.

### DETAILED DESCRIPTION

One embodiment of the current disclosure used retrospective data to computationally model the personalized absolute risk reduction from ADT added to RT in males with prostate cancer who had a CCR score. As personalized risk estimators become more widely available, it is unclear how patient-level precision medicine approaches can be reconciled with population-level therapeutic options. In some embodiments, this disclosure models the personalized absolute risk reduction of adding ADT to RT for prostate cancer using a relative risk reduction from prospective randomized trials. In some embodiments, the CCR score provides personalized risk information, and the model showed that those with scores above the Prolaris multi-modal treatment threshold benefit more from combined treatment. In some embodiments, the model revealed that adding ADT to RT would prevent metastasis within 10 years for 1 in 12 individuals with above-threshold scores, whereas only 1 in 116 individuals with below-threshold scores would benefit from the treatment.

Kishan et al., (Lancet Oncol. 2022; 23(2):304-316) performed an individual patient-level meta-analysis that quantified the relative benefit of adding ADT to RT for men across a broad spectrum of NCCN risk groups. They found that adding ADT to RT reduces metastasis risk regardless of risk group, age, and radiotherapy dose delivered. However, since the magnitude of the benefit could vary, they recommended shared decision-making between doctors and patients, given the potential toxicity of ADT.

To address this critical need, embodiments described herein quantified how adding ADT to RT would reduce metastasis risk at the individual patient level by applying Kishan's findings to the personalized risk estimates derived from the CCR score, which combines molecular and clinical prognostic information. The results of the current disclosure revealed that CCR score can inform the personalized absolute risk reduction due to treatment with ADT, with reduced 10-year risk of metastasis predicted in men above the multimodality threshold when ADT is added to RT.

The methodology could be applied to any validated nomogram or biomarker that produces an individual risk estimate for metastasis. However, the nomogram or biomarker risk estimates must be calibrated exclusively on a population treated with RT without ADT.

In some embodiments, personalized risk estimates can address inherent issues with the population-based risk cohorts used by guidelines or as clinical trial criteria. These cohorts represent a broad spectrum of actual risk, with patients who may experience vastly different levels of absolute risk reduction. For example, data from RTOG 0126 evaluated dose escalation using RT alone in patients with D'Amico/NCCN-classified intermediate-risk disease (Moughan et al., JAMA Oncology. 2018; 4(6):e180039; D'Amico et al., Jama. 1998; 280(11):969-974). At the trial population level, patients enrolled in RTOG 0126 had a 95% CI for 10-year risk of metastasis between 3% and 9%. Validated personalized risk estimators based on CCR scores of men with intermediate-risk disease in the clinically tested cohort placed the median individual risk at 4.6%, within the RTOG 0126 range. However, individuals categorized as having unfavorable intermediate disease had patient-level estimated risks that varied between 0.2% and 67.9% when using the CCR score as a personalized risk estimator, and high-risk individuals varied from 0.5% to >99% (Table 1). Assuming a 41% relative ADT benefit, the average absolute risk reduction was 2.63% in unfavorable intermediate-risk individuals and 7.06% in high-risk individuals, corresponding to respective NNTs of 38 and 14 (Table 2). This result suggests that those who are recommended to receive RT+ADT based on current guidelines may experience a range of benefits from RT+ADT, thereby highlighting the importance of considering individual risk factors when making treatment decisions.

TABLE 1
Distributions of estimated 10-year risk of metastasis given single-mode RT in
the Clinical Cohort, summarized by NCCN risk group and in the full cohort.

| NCCN risk | | | 5th | 25th | | 75th | 95th | |
|---|---|---|---|---|---|---|---|---|
| group | N | Min. | quantile | quantile | Median | quantile | quantile | Max. |
| Low | 19013 | 0.1% | 0.3% | 0.5% | 0.7% | 1.1% | 2.1% | 28.3% |
| Favorable | 16812 | 0.2% | 0.6% | 1.0% | 1.5% | 2.3% | 4.3% | 29.5% |
| Intermediate | | | | | | | | |
| Unfavorable | 14453 | 0.2% | 1.3% | 2.6% | 4.6% | 8.6% | 19.6% | 67.9% |
| Intermediate | | | | | | | | |
| High | 6207 | 0.5% | 2.6% | 6.9% | 13.8% | 28.9% | 66.2% | >99% |
| Overall | 56485 | 0.1% | 0.4% | 0.9% | 1.7% | 4.3% | 20.8% | >99% |
| Cohort | | | | | | | | |

TABLE 2
Mean absolute risk reduction from ADT and corresponding NNT
for patients above and below the multimodality threshold assuming
different overall relative benefits of ADT (32%, 41%, and
48%) and interaction strengths (0%, 50%, and 100%).

| | Below | | Above | |
|---|---|---|---|---|
| | Multimodality | | Multimodality | |
| | Threshold | | threshold | |
| | Absolute | | Absolute | |
| | Risk | | Risk | |
| | Reduction | NNT | Reduction | NNT |
| 32% Relative | | | | |
| Benefit of ADT | | | | |
| 0% relative interaction (no | 0.67% | 149 | 6.25% | 16 |
| interaction) | | | | |
| 50% relative interaction | 0.71% | 141 | 8.79% | 11 |
| 100% relative interaction | 0.74% | 135 | 11.06% | 9 |
| (maximum interaction) | | | | |
| 41% Relative | | | | |
| Benefit of ADT | | | | |
| 0% relative interaction (no | 0.86% | 116 | 8.19% | 12 |
| interaction) | | | | |
| 50% relative interaction | 0.90% | 111 | 11.30% | 9 |
| 100% relative interaction | 0.94% | 107 | 13.92% | 7 |
| (maximum interaction) | | | | |
| 48% Relative | | | | |
| Benefit of ADT | | | | |
| 0% relative interaction (no | 1.01% | 99 | 9.75% | 10 |
| interaction) | | | | |
| 50% relative interaction | 1.06% | 94 | 13.24% | 8 |
| 100% relative interaction | 1.09% | 92 | 15.98% | 6 |
| (maximum interaction) | | | | |

Using this methodology, we can inform patients of the personalized absolute risk reduction from adding ADT to RT when contemplating treatment intensification. This absolute risk reduction could also be easily translated into an NNT, the number of patients needed to treat with ADT to prevent metastasis in one patient. When polled, expert physicians treating genitourinary malignancies and the broader public have unpredictable individual thresholds for whether they would add ADT to RT, assuming they were choosing for themselves. Some would accept the therapy if the personalized absolute risk reduction at 10 years were 1%, whereas others would need a threshold of at least 10% before accepting RT+ADT over RT alone. For most persons, the absolute risk reduction would have to exceed 4%. This value is extremely close to the risk reduction at the multimodality threshold proposed in prior works utilizing the CCR score. In this study, men below the multimodality threshold had an average absolute risk reduction of only 0.86%, whereas those above had an average absolute risk reduction of 8.2%. This average absolute risk reduction observed in those above the multi-modal treatment threshold was even higher than the 7.06% average absolute risk reduction observed in the NCCN high-risk subset of the cohort, demonstrating the utility of the CCR score in predicting the benefit of adding ADT to RT.

The benefits of the presently disclosed methods are that the personalized risks estimated by the biomarker (CCR score) can be calibrated exclusively in a patient population treated with RT alone and can determine absolute risk reduction from adding ADT to RT based on evidence from prospectively randomized trials. The limitations are that the risk estimates with RT alone are generally derived from retrospective datasets, although the RT-Alone Cohort used to build the estimates may have similar clinical characteristics and produce similar risk estimates to a prospective cohort.

The CCR score gives a precise and personalized risk estimate of metastasis calibrated in a population treated with RT alone. Therefore, a patient and their physician considering RT for localized prostate cancer would know precisely how intensifying treatment with ADT might affect their oncologic outcomes when weighed against its toxicities. By understanding the absolute risk reduction and NNT for their case, patients can make a risk-based assessment compatible with their personal goals instead of a strictly guideline-concordant recommendation predicated on expert physician judgment.

### EXAMPLES

### Example 1—Data Sets

Male patients diagnosed with prostate cancer who underwent Prolaris testing (Myriad Genetics, Salt Lake City, UT) either clinically or retrospectively through research studies. The Single-Mode RT Cohort includes patients treated with single-mode RT across two retrospectively tested, previously published cohorts (N=467). Additional details on patient and specimen characteristics, inclusion and exclusion criteria, any treatments received, patient follow-up, and the period from which cases were collected are described, as applicable, in previous publications. The Clinical Cohort includes patients with a clinical Prolaris biopsy test reported between Jan. 1, 2020, and Oct. 31, 2022 (N=56,485). Patients were untreated at the time of the biopsy used for testing, and additional clinical follow-up, including treatment choices, is unknown. Test reports were limited to the first reported test per patient with calculable mortality and metastasis risk. Any tests issued to patients under 18 years of age at the time of testing were excluded. Summaries of the clinical and molecular characteristics of the cohorts are included in Table 3.

TABLE 3
Clinical and molecular characteristics of men with prostate cancer from
the Single-Mode RT Cohort (N = 467) and the Clinical Cohort (N = 56,485).

| | Single-Mode RT Cohort | | Clinical Cohort | |
|---|---|---|---|---|
| | N = 467 | | N = 56,485 | |
| | N (%) or Median (IQR) | | N (%) or Median (IQR) | |
| Patient age at diagnosis | 70 | (64, 76) | 67 | (62, 72) |
| Gleason Score | | | | |
| <7 | 68 | (14.6%) | 22216 | (39.3%) |
| 3 + 4 | 263 | (56.3%) | 21962 | (38.9%) |
| 4 + 3 | 110 | (23.6%) | 7797 | (13.8%) |
| >7 | 26 | (5.6%) | 4510 | (8.0%) |
| Percent positive cores | 33.3% | (21.4%, 50.0%) | 25.0% | (13.0%, 41.0%) |
| Clinical T stage | | | | |
| T1 | 285 | (61.0%) | 50437 | (89.3%) |
| T2 | 172 | (36.8%) | 5898 | (10.4%) |
| T3 | 10 | (2.1%) | 150 | (0.3%) |
| Diagnostic PSA | 7.0 | (5.0, 11.1) | 6.2 | (4.8, 8.2) |
| CAPRA | 4 | (3, 5) | 3 | (2, 4) |
| NCCN | | | | |
| Very Low/Low Risk | 0 | (0%) | 19013 | (33.7%) |
| Favorable Intermediate Risk | 172 | (36.8%) | 16812 | (29.8%) |
| Unfavorable Intermediate Risk | 254 | (54.4%) | 14453 | (25.6%) |
| High/Very High Risk | 41 | (8.8%) | 6207 | (11.0%) |
| CCP Score | 0.3 | (−0.2, 0.8) | −0.5 | (−1.0, 0.0) |
| CCR Score | 1.617 | (1.122, 2.178) | 0.771 | (0.258, 1.503) |
| Below AS Threshold | 56 | (12.0%) | 28859 | (51.1%) |
| Between Thresholds | 285 | (61.0%) | 20435 | (36.2%) |
| Above MM Threshold | 126 | (27.0%) | 7191 | (12.7%) |

There were no missing values for demographic, clinical, or molecular data.

### Example 2—CCR Score

CCP scores were calculated and were blinded to patient outcome, as described previously. RNA extraction was performed using either miRNeasy (Qiagen, Hilden, Germany) or MagMAX (Thermo Fisher Scientific. The CCP score was calculated as the average expression of normalized CCP genes that were amplified using either TaqMan Low-Density Arrays (Applied Biosystems, Foster City, CA) or OpenArrays (Thermo Fisher Scientific) were used to amplify 31 previously defined CCP genes (FOAM1, CDC20, CDKN3, CDC2, KIFI1, KIAA0101, NUSAP1, CENPF, ASPM, BUB1B, RRM2, DLGAP5, BIRC5, KIF20A, PLK1, TOP2A, TK1, PBK, ASF1B, C18orf24, RAD54L, PTTG1, CDCA3, MCM10, PRC1, DTL, CEP55, RAD51, CENPM, CDCA8, and ORC6L) normalized to 15 housekeeper genes. The CCP score was calculated as the average expression of normalized CCP genes. After measuring RNA expression levels, the CCP molecular score was combined with the University of California, San Francisco Cancer of the Prostate Risk Assessment (CAPRA) score to produce the predefined CCR score [(0.39×CAPRA)+(0.57×CCP)]. A CCR score of 2.112 has been validated as a prognostic threshold for determining the benefit of multimodal therapy. The CCR score can be translated into a personalized risk estimate of 10-year metastasis.

### Example 3—Statistical Methods

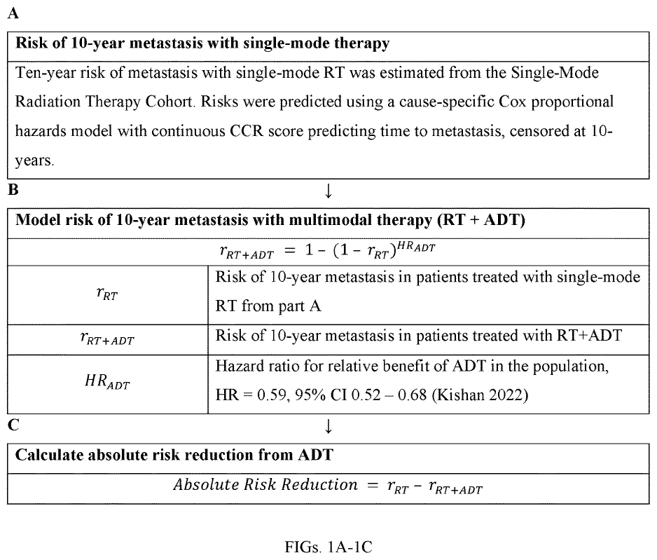

The ability of the CCR score to provide an estimate of absolute risk reduction from ADT added to RT was modeled based on previous reports, as shown in FIG. 1. First, the 10-year risk of metastasis with single-mode RT was modeled as a function of CCR score, r_{RT}(CCR), in the Single-Mode RT Cohort (FIG. 1A). The 10-year risk of metastasis in patients treated with RT+ADT was estimated by introducing a relative ADT benefit, HR_{ADT}, to the risk with single-mode RT according to a proportional hazards model (FIG. 1B).

r_{RT+ADT}=1−(1−r_{RT})^{HRADT} Equation 1:

HR_{ADT} is the hazard ratio for the benefit of ADT in the population, as modeled in The Meta-Analysis of Randomized Trials in Cancer of the Prostate (MARCAP), which utilized several prospectively randomized trials of RT alone compared to RT+ADT.²³ In this meta-analysis, the overall reduction in distant metastasis was estimated to be 41%, with a 95% confidence interval (CI) of 32% to 48%²³ (HR_{ADT}=0.59, 95% CI: 0.52-0.68).

The absolute risk reduction of ADT added to RT was determined according to Equation 2 (FIG. 1C).

Absolute risk reduction=r_{RT}−r_{RT+ADT} Equation 2:

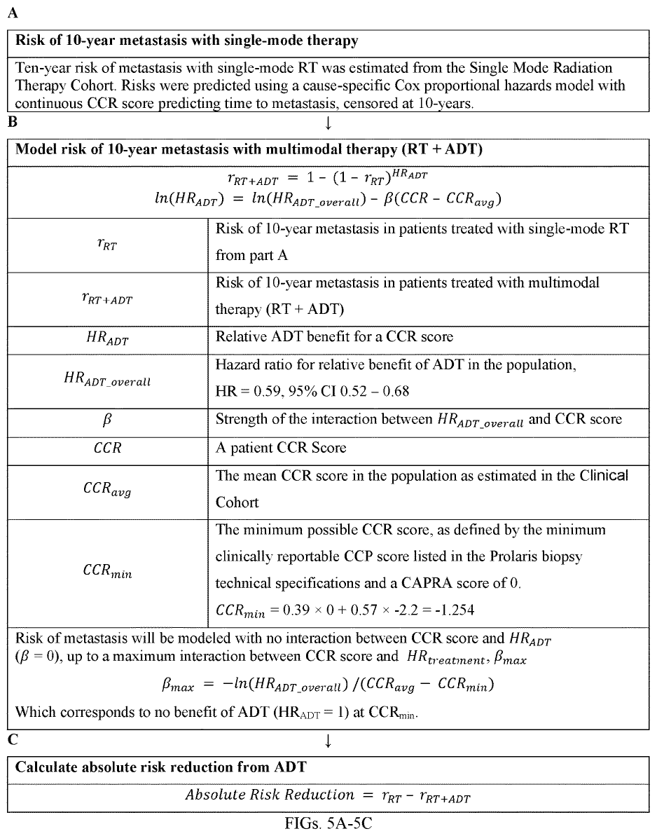

While there is no evidence of any dependence of the relative benefit of ADT (HR_{ADT}) on CCR scores, patients with higher CCR scores may benefit more from treatment with ADT than patients with low CCR scores. As a sensitivity analysis, a simple dependence of the relative benefit of ADT on CCR was introduced into the calculations as an interaction between the relative benefit of ADT and CCR (FIGS. 5A-5C).

CIs were generated for the estimated r_{RT+ADT} and absolute risk reduction through simulation. To do so, the risk for a given CCR score was randomly generated from a normal distribution centered at log(−log(r_{RT})) with the standard deviation equal to the standard error of log(−log(r_{RT})). HR_{ADT} was randomly generated from a normal distribution centered at log(HR_{ADT}) with the standard deviation equal to the half-width of the CI on the log scale. The sampled r_{RT} and HR_{ADT} were then used to generate r_{RT+ADT}, as described in Equation 1. When this simulation is repeated many times for each CCR score, the values at the 2.5′ and 970.5^{th} quantiles can be taken as 95% CIs for the estimated value of r_{RT+ADT} for that CCR score. The difference between the randomly sampled r_{RT} and the r_{RT+ADT} values, calculated using the randomly sampled r_{RT} value, is the absolute risk reduction. The 2.5^{th} and 97.5^{th} quantiles of the simulated distribution (comprised of 3 million simulations) for the absolute risk reduction for each CCR score were smoothed using Loess regression and taken as the 95% CIs for the absolute reduction in risk due to treatment with ADT for that CCR score.

The NNT was calculated as one divided by the absolute risk reduction and rounded to the nearest whole number.

The distribution of CCR scores in the Clinical Cohort was used as an approximation of the distribution of CCR scores in the general population of prostate cancer patients. The r_{RT}, r_{RT+ADT}, and absolute risk reduction were calculated for each patient in the Clinical Cohort.

### Example 4—Results

The 10-year risk of metastasis with single-mode RT (r_{RT}) was calculated in the Single-Mode RT Cohort (N=467). Patients in the Single-Mode RT Cohort had a distribution of CCR scores with a 5th percentile and a 95th percentile of 3.072 (corresponding 10-year risk of metastasis with single mode RT=1.2% to 28.7%), and included National Comprehensive Cancer Network® (NCCN) favorable intermediate-risk, unfavorable intermediate-risk, and high-risk patients. More than a quarter (27.0%) of patients had CCR scores above the multimodality threshold (Table 3).

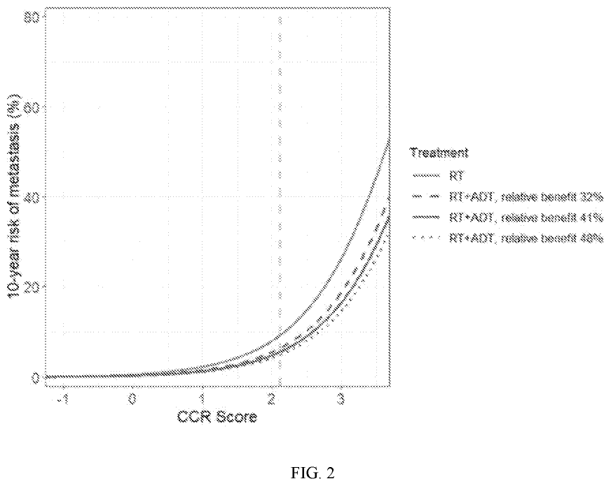

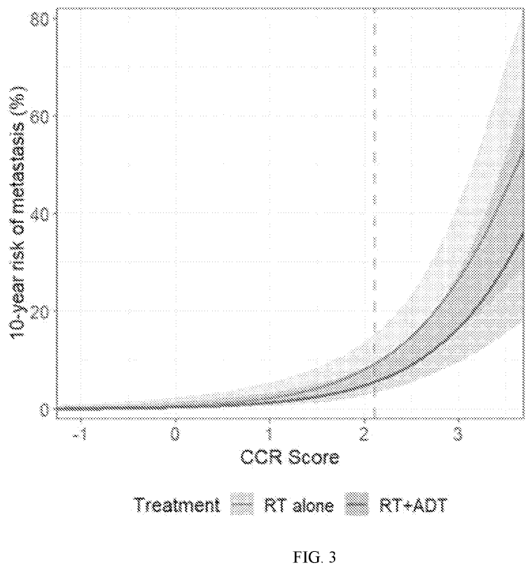

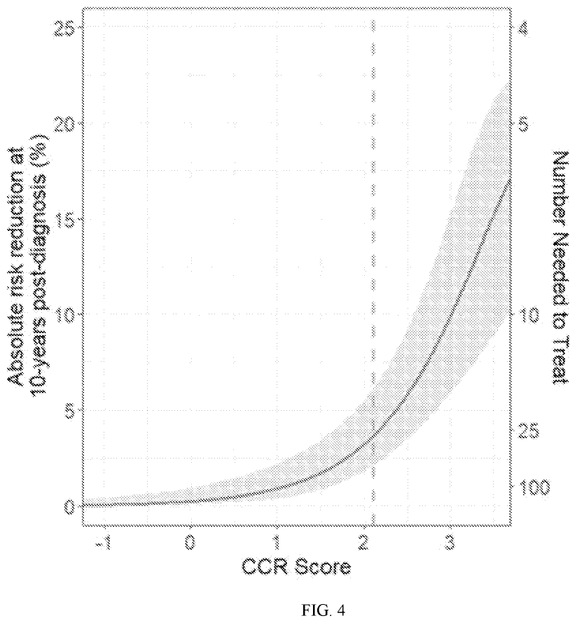

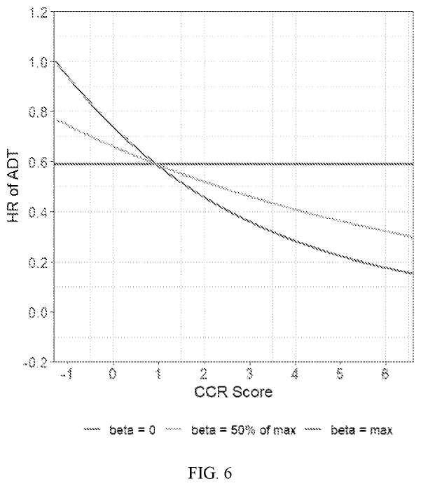

The risk of metastasis with RT+ADT (r_{RT}-ADT) was calculated using r_{RT}, and a relative benefit from ADT of 32%, 41%, and 48%, corresponding to the estimate and 95% confidence limits of HR_{ADT} published in Kishan 2022 (FIG. 2). The estimated risk of metastasis with RT+ADT, assuming a relative benefit from ADT of 41%, is shown with simulated CIs in FIG. 3. The corresponding absolute risk reduction and NNT are shown in FIG. 4. The absolute reduction in the risk of metastasis from ADT was low at low CCR scores and increased to 17.1% at CCR=3.690 (FIG. 4). At very high CCR scores, the CIs for the estimated risk with RT alone became wider, making it difficult to accurately estimate the risk reduction with RT+ADT. Consequently, the risk and absolute risk reduction were plotted for CCR scores below the 99th percentile of clinically tested patients (CCR<3.690). For patients with CCR scores above the 99th percentile (CCR≥3.690), the risk of metastasis with RT alone that is high enough to always warrant consideration of multi-modal treatment (FIG. 6). Therefore, a personalized absolute risk reduction estimate is not expected to provide clinically meaningful treatment guidance for patients with very high CCR scores.

The 10-year risk of metastasis given single-mode RT (r_{RT}) at the multimodality threshold (CCR=2.112) is 9.2% (95% CI 5.4%-15.3%). Assuming a relative benefit of ADT of 41%, the risk at the multimodality threshold given RT+ADT (r_{RT+ADT}) was 5.5% (95% CI 3.2%-9.5%), corresponding to an absolute risk reduction from ADT of 3.7% (95% CI 2.1%-6.1%; NNT=27, 95% CI 16-48) (FIG. 4).

The risk estimates across varying levels of the relative benefit of ADT were calculated for the Clinical Cohort (N=56,485). The distribution of CCR scores had a 5th percentile of −0.351 and 95th percentile of 2.787, spanning all NCCN risk categories. Most patients in the Clinical Cohort had CCR scores at or below the multimodality threshold (87.3%), whereas only 12.7% had CCR scores above the multimodality threshold (Table 3). The 10-year risk of metastasis assuming single-mode RT was calculated for each patient in the Clinical Cohort. The risk of metastasis was summarized for the full cohort, as well as for each NCCN risk group in Table 1. In NCCN unfavorable intermediate-risk patients, the 5th and 95th percentiles of individualized risk of metastasis with RT alone were 1.3% and 19.6%, respectively. The 5th and 95th percentiles of risk in NCCN high-risk patients were 2.6% and 66.2%, respectively. These distributions indicated that the risk of metastasis varied greatly, even within population-based risk groups (Table 2). The 10-year risk of metastasis given RT+ADT was also estimated for each patient, and the personalized absolute risk reduction from ADT added to RT was calculated. Assuming a relative benefit of ADT of 41%, the average absolute risk reduction below the multimodality threshold was 0.86% (NNT=116), whereas the average absolute risk reduction above the multimodality threshold was 8.19% (NNT=12). A summary of the average absolute risk reductions and corresponding NNT values assuming relative benefits of 32%, 41%, and 48% are shown by multimodality threshold status in Table 4. Summaries of the average absolute risk reduction and NNT values by NCCN category are shown in Table 2.

TABLE 4
Mean, 5^{th}, and 95^{th} percentiles of the distribution of absolute
risk reduction from ADT added to RT in the Clinical Cohort
for patients above and below the multimodality threshold,
assuming different overall relative benefits of ADT (32%,
41%, and 48%), and with corresponding NNT.

| | Below Multimodality | | Above Multimodality | |
|---|---|---|---|---|
| | Threshold | | threshold | |
| | Mean (5^{th} | | Mean (5^{th} | |
| | percentile, 95^{th} | | percentile, 95^{th} | |
| | percentile) | | percentile) | |
| | Absolute | | Absolute | |
| | Risk | | Risk | |
| | Reduction | NNT | Reduction | NNT |
| 32% Relative | 0.67% (0.12%, | 149 (819, | 6.25% (3.05%, | 16 (33, 8) |
| Benefit of ADT | 2.06%) | 49) | 13.2%) | |
| 41% Relative | 0.86% (0.16%, | 116 (639, | 8.19% (3.92%, | 12 (25, 6) |
| Benefit of ADT | 2.64%) | 38) | 17.9%) | |
| 48% Relative | 1.01% (0.18%, | 99 (546, | 9.75% (4.61%, | 10 (22, 5) |
| Benefit of ADT | 3.10%) | 32) | 21.8%) | |

Only 8.8% of the RT-Alone Cohort had NCCN high-risk or very high-risk prostate cancer; therefore, decisions to limit ADT in this subgroup should be approached with caution. The retrospective dataset also produced wide CIs for the top 1% of clinically observed CCR scores. Additionally, the analysis assumes that the relationship between CCR score and ADT responsiveness is constant. It is possible that the benefit of ADT varies with the value of the biomarker. However, this effect can be modelled by including the interaction between ADT and CCR, which indicates that biomarker-dependent effects.

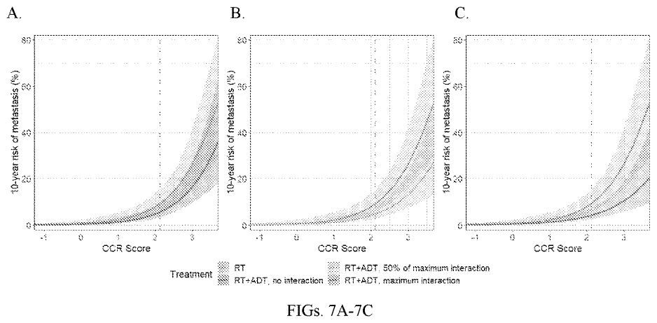

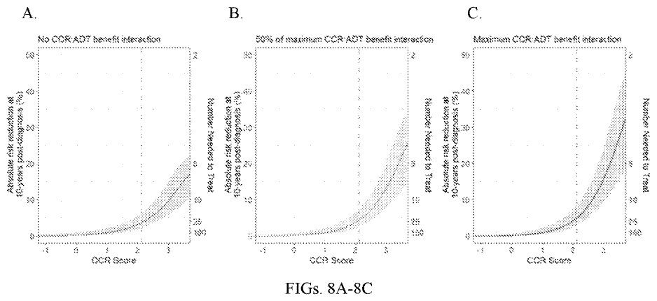

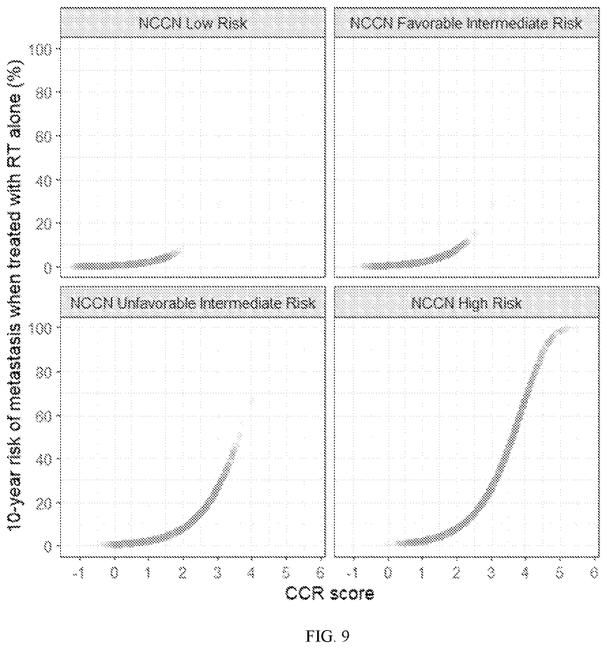

As a sensitivity analysis, the dependence of the relative benefit of ADT on CCR was introduced into the calculations as an interaction between the relative benefit of ADT and CCR. The maximal interaction strength was calculated assuming a patient's relative benefit from ADT was 0 at the lowest possible CCR score while maintaining the overall HR_{ADT} value at 0.59. HR_{ADT} as a function of the CCR score was then calculated with the maximal interaction strength and 50% maximal interaction strength (FIG. 7A-7C). For higher CCR scores, the r_{RT+ADT} values calculated with these RT+ADT interaction strengths were lower than those calculated in the absence of an interaction. For lower CCR scores, there was no substantial difference when the RT+ADT interactions were added to the calculation (FIGS. 8A-8C). Although the presence of an interaction resulted in more extreme differences in absolute risk reduction and NNT by multimodality threshold status, the results were not substantially different than when conservatively assuming no interaction between the relative benefit of ADT and CCR score (FIG. 9, Table 2).

Finally, the impact of race or ancestry on estimates of the risk and benefit of ADT were not explored in the dataset of the present disclosure or the meta-analysis used to estimate the relative benefit of ADT utilized in this study. It is unclear how the score's performance may vary across different demographic groups which may limit its utility and accuracy. However, the CCP score has been tested in African American populations, where it is demonstrated that African American men have similar prostate cancer outcomes to non-African American patients after accounting for CCP and clinicopathologic variables. External validation using additional independent datasets could continue to refine the model, and potentially be able to account for the effect different patient populations, treatment settings, and healthcare systems.

### Example 5—Predicting Absolute Risk Reduction (AAR)

Method: The clinical cell-cycle risk (CCR) score combines the University of California, San Francisco's Cancer of the Prostate Risk Assessment and the cell cycle progression molecular score to accurately assess prostate cancer aggressiveness. The effect of ADT added to RT was modeled using a 10-year risk of metastasis as a function of continuous CCR score for patients treated with RT alone. The relative benefit of ADT added to RT to reduce distant metastasis was modeled using published data from The Meta-Analysis of Randomized trials in Cancer of the Prostate (MARCAP) which utilized several prospectively randomized RT±ADT trials.² The average absolute benefit was calculated for patients with CCR scores above and below a prespecified multimodality treatment threshold (MTT) using the distribution of scores in a cohort of patients clinically tested by Myriad Genetics

Results: The clinically tested cohort consisted of 56,485 patients spanning all National Comprehensive Cancer Network risk categories (33.7% very low/low risk, 29.8% favorable intermediate risk, 25.6% unfavorable intermediate risk, 11.0% high/very high risk), with 87.2% having CCR scores at or below the MTT and 12.7% with CCR scores above the threshold. The average absolute benefit of ADT treatment was 0.86% (NNT=116) for patients with CCR scores below the MTT and 8.2% (NNT=12) for patients with CCR scores above the MTT. Patients with CCR scores at precisely the MTT had a predicted absolute benefit of 3.7% (NNT=27). Thus, predicting an individual's benefit of adding ADT to RT, and the risk threshold is consistent with patient attitudes about when to use or omit ADT.

1. A method of determining an absolute risk reduction (AAR) of disease progression in a subject, comprising:
(a) obtaining a biological sample from a subject;
(b) calculating a clinical cell-cycle risk (CCR) score (r_{RT}) for treating the subject with radiation therapy (RT) alone;
(c) calculating a multimodal risk score (r_{RT+ADT}) for treating the subject with a multimodmodal therapy comprising androgen deprivation therapy (ADT) and RT; and
(d) calculating the AAR (r_{RT}−r_{RT+ADT}) for the subject.

2. The method of claim 1, wherein calculating the CCR score comprises combining a cancer of the prostate risk assessment (CAPRA) score and a cell-cycle progression (CCP) score.

3. The method of claim 1, wherein the CCP score is calculated as the average expression of 31 CCP genes normalized to 15 housekeeping genes.

4. The method claim 1, wherein the multimodal risk score (r_{RT+ADT}) is calculated according to r_{RT+ADT}=1−(1−r_{RT})^{HR}_{ADT}, wherein HR_{ADT} is a relative ADT benefit as a hazard ratio.

5. The method of claim 1, wherein the AAR is calculated based on a combination treatment of ADT and RT compared to RT alone.

6. The method of claim 1, wherein the subject has been diagnosed with prostate cancer.

7. The method of claim 1, wherein the disease progression is metastasis.

8. The method of claim 1, wherein the clinical cell-cycle risk (CCR) score (r_{RT}) is a risk estimate of developing metastasis after treatment with RT alone.

9. The method of claim 1, wherein the multimodal risk score (r_{RT+ADT}) is a risk estimate of developing metastasis after treatment with ADT and RT in combination.

10. The method of claim 1, further comprising:
(e) comparing the AAR to a multimodal treatment threshold (MTT), wherein an AAR over the MTT would be predictive of lower risk of metastasis and an AAR under the MTT would be predictive of a higher risk of MTT in subject receiving ADT and RT.
